SEQUENCE LISTING (10 sequences)

SEQ ID NO: 1 - DNA, 20 bp, Artificial Sequence
  Synthetically generated oligonucleotide
gggggacgatcgtcgggggg

SEQ ID NO: 2 - DNA, 24 bp, Artificial Sequence
  Synthetically generated oligonucleotide
tcgtcgttttgtcgttttgtcgtt

SEQ ID NO: 3 - DNA, 25 bp, Artificial Sequence
  Synthetically generated oligonucleotide
tcgtcgtcgttcgaacgacgttgat

SEQ ID NO: 4 - DNA, 20 bp, Artificial Sequence
  Synthetically generated oligonucleotide
gggggagcatgctggggggc

SEQ ID NO: 5 - DNA, 24 bp, Artificial Sequence
  Synthetically generated oligonucleotide
tgctgcttttgtgcttttgtgctt

SEQ ID NO: 6 - DNA, 25 bp, Artificial Sequence
  Synthetically generated oligonucleotide
tgctgctgcttgcaagcagcttgat

SEQ ID NO: 7 - DNA, 25 bp, Artificial Sequence
  Synthetically generated oligonucleotide
tcgtcgtcgttcgaacgacgttgat

SEQ ID NO: 8 - DNA, 25 bp, Artificial Sequence
  Synthetically generated oligonucleotide
tcgtcgtcgttcgaacgacgttgat

SEQ ID NO: 9 - DNA, 25 bp, Artificial Sequence
  Synthetically generated oligonucleotide
tcgtcgtcgttcgaacgacgttgat

SEQ ID NO: 10 - DNA, 25 bp, Artificial Sequence
  Synthetically generated oligonucleotide
tcgtcgtcgttcgaacgacgttgat

(19) United States
(12) Patent Application Publication
(10) Pub. No.: US 2009/0191185 A1
(43) Pub. Date: Jul. 30, 2009

(54) Reducing Cancer Cell Invasion Using an Inhibitor of Toll Like Receptor Signaling

(75) Inventor: Katri Selander, Birmingham, AL (US)

Correspondence Address: FISH & RICHARDSON P.C., P.O BOX 1022, Minneapolis, MN, 55440-1022, US

(73) Assignee: THE UAB RESEARCH FOUNDATION, Birmingham, AL (US)

(21) Appl. No.: 12/298,469

(22) PCT Filed: Apr. 13, 2007
(86) PCT No.: PCT/US2007/066589

§ 371 Date: Nov. 12, 2008

Related U.S. Application Data
(60) Provisional application No. 60/745,694, filed on Apr. 26, 2006.

(51) Int. Cl. A61K 39/395; A61K 31/4706; A61K 31/335; A61K 31/35; A61K 31/7088; C12Q 1/68; C12Q 1/02

(52) U.S. Cl. 424/130.1; 514/313; 514/297; 514/450; 514/453; 514/44; 435/6; 435/29

(57) ABSTRACT

Provided herein are methods and compositions for reducing the invasiveness of cancer cells. Such methods and compositions are particularly useful for cancer cells that express a member of the Toll Like Receptor9 (TLR9) subfamily and are useful in selecting the proper treatment for a subject with cancer.

### CROSS-REFERENCE TO RELATED APPLICATIONS

This application claims the benefit of U.S. Provisional Application No. 60/745,694 filed Apr. 26, 2006.

### STATEMENT REGARDING FEDERALLY FUNDED RESEARCH

This invention was forded by the Department of Defense, Grant No. W81XWH-04-1-0600. The United States Government may have certain rights in this invention.

### TECHNICAL FIELD

The present application relates to methods and compositions for the treatment of cancer.

### BACKGROUND

Environmental and epigenetic factors, such as infections and resulting inflammation are important regulators of tumor progression. The innate immune system can promote tumor development and progression through inflammation-dependent mechanisms. For example, chemokines and cytokines derived from the immune and inflammatory cells can dramatically affect the host microenvironment and cancer cell behavior, resulting in increased growth and metastasis. Increased growth and metastasis have a profound influence on morbidity and mortality in subjects with cancer. Thus, needed in the art are compositions and methods for reducing the abilities of environmental and epigenetic factors to increase the invasive capacity of cancer cells.

### SUMMARY

Provided herein are methods and compositions for reducing the invasiveness of cancer cells. Such methods and compositions are particularly useful for cancer cells that express a member of the Toll Like Receptor 9 (TLR9) subfamily and are useful in selecting the proper treatment for a subject with cancer.

The details of one or more embodiments are set forth in the accompanying drawings and the description below. Other features, objects, and advantages will be apparent from the description and drawings, and from the claims.

### BRIEF DESCRIPTION OF DRAWINGS

FIG. 1 shows that human breast cancer cell lines exhibit, different levels of TLR9 expression. FIG. 1A shows the expression profile of the mRNAs for various TLRs as studied with a DNA-array in MDA-MB-231 cells. The calculated, numeric levels of expression of each TLR mRNA were obtained after blank subtraction and correction for the expression level of actin. FIGS. 1B and 1C show specific expression of the TLR9 protein as detected in permeabilized MDA-MB-231 cells, using PE-conjugated anti-TLR9 antibody in flow cytometry (1B) and immunohistochemistry (1C), for which omission of the primary antibody served as a negative control. FIG. 1D shows Western blot detection of the TLR9 protein in the various human breast cancer cells (upper panels). The same blots were stripped and re-blotted with anti-actin antibodies, to show equal loading of total protein.

FIG. 2 shows TLR9 expression in human breast cancer tissues. FIG. 2A shows a Western blot with the expression of TLR9 in MDA-MB-231 cells and in normal breast tissue obtained at mammoplasty and in breast cancer specimens lanes 1-5. FIG. 2B shows TLR9 expression in immortalized MCF-10A mammary epithelial cells. Both blots were stripped and reblotted with, anti-actin antibody to show that the differences in TLR9 expression are not due to unequal loading of total protein.

FIG. 3 shows TLR9 agonistic CpG-motif containing unmethylated oligonucleotides (CpG-ODNs) induce invasion of TLR9 expressing cancer cells in vitro. FIG. 3A are images of MDA-MB-231 cells (arrows) that have invaded through the Matrigel-membranes during 18 hours of invasion in response to vehicle control or to Type C CpG-ODN, which is a TLR9 agonist. FIG. 3B shows quantitation of the effects of the various CpG-ODNs on the invasive capacity of MDA-MB-231 cells as studied in Matrigel-assays. Data represent the fold-increase in the number of invaded cells, as compared with vehicle controls (dotted line, set to 1) in each group. Mean±sd, n=4, ** p<0.01, *** p<0.001 vs. vehicle. FIG. 3C shows MDA-MB-231 cells cultured for 7 days on 3-dimensional collagen cultures in the presence of vehicle or 10 μM CpG-ODNs. The arrows indicate the front of the invading cells in the gels after they were prepared into H&E-stained histological samples. FIG. 3D shows the numbers of invading cells, or the depths of the invasion front as counted or measured from 5 representative sites in the cut sections in FIG. 3C. Mean±sd, n=3, representing the number of sections viewed in each group, * p<0.05, *** p<0,001 vs. vehicle. FIG. 3E is a Western blot of the TLR9 protein in U373 astrocytoma and in D54MG glioblastoma cells (upper panels), where MCF-7cells represent a negative control. The same blots were stripped and reblotted with anti-actin antibodies (lower panels), to show equal loading. FIG. 3F shows the effects of 10 μM type C CpG-ODNs on the invasive capacity of the indicated cells were studied in Matrigel-assays. Data represent the fold-increase in the number of invaded cells, as compared with vehicle controls (dotted line) in each group. Mean±sd, n=4, ** p<0.01, *** p<0.001 vs. vehicle. FIG. 3G shows that treatment with 10μM type C CpG-ODNs or non-CpG-ODN induced a significant invasion response in the MCF-10A cells, but they had no significant effect on the poorly invasive, TLR9-MCF-7 breast cancer cells. Data are expressed as mean±sd, n=3-4, * p<0.05, ** p<0.01 vs. basal.

FIG. 4A shows the effect on the viability of the indicated breast cancer cells as tested with MTS-assays, after treatment for 24 hours with 10 μM type C CpG-ODNs or with vehicle. Data represent viability as a percentage of vehicle control, mean±sd, n=4. FIGS. 4B, 4C and 4D show the effect of type C CpG-ODN or non-CpG-ODN on the long-term viability of MDA-MB-231 cells (4B), T47-D cells (4C) or MCF-7 cells (4D) as studied with MTS-assays. Mean±sd, n=5, *** p<0.001 vs. PBS-control.

FIG. 5 shows CpG-ODN-induced invasion is blocked with chloroquine. MDA-MB-231 cells were allowed to invade through Matrigel-membranes for 18 hours in the presence of type C CpG-ODN or non-CpG-ODN (10 μM) with vehicle or chloroquine (10 μM). Data are expressed as fold-increase in invasion, as compared with the corresponding unstimulated group. Mean±sd, n=3, ** p< 0.01 vs. vehicle-control.

FIG. 6 shows that CpG-ODN-treatment increases MMP-activity in MDA-MB-231 cells. FIG. 6A shows supernatants from CpG-ODN-treated MDA-MB-231 cells run on 30% gelatin gels. Treatment with type C CpG-ODNs resulted in the appearance of a gelatinolytic band of about 50 kDA (arrow), which did not disappear in the presence of aprotinin but which was abolished by the addition of a global MMP-inhibitor, GM6001, to the final incubation. The formed band was of a similar size than that induced by a positive control for MMP-13 (about 50 kDa, arrows). FIG. 6B shows that the MMP-inhibitor, but not aprotinin, (both at 2 μM) inhibited CpG-ODN-induced invasion. Data represent the number of invaded cells as a percentage of the type C CpG-induced (10 μM) control for each group. Mean±sd, n=3, *** p<0,001 vs. CpG-ODN-treatment alone.

FIG. 7 shows that CpG-ODN induced invasion is mediated via MMP-13. FIG. 7A shows levels of active MMP-13 from the supernatants of vehicle or type C CpG-ODN-treated (10 μM) in MDA-MB-231/T47-D and MCF-7 breast cancer cells, as analyzed with ELISA. Data are expressed as fold-increase, as compared with the vehicle-treated controls for each cell line. Mean±sd, n=3-4, *** p<0.001 vs. vehicle. FIG. 7B shows the invasive capacity of MDA-MB-231 and T47-D cells as investigated in Matrigels in the presence of 10 μM type C CpG-ODNs with neutralizing antibody against MMP-13 or with a control IgG antibody. Data represent the number of invaded cells as a % of control IgG-treated group. Mean±sd, n=3, * p<0.05 vs. IgG-treated group. FIG. 7C shows the % of active MMP-8 in MDA-MB-231 cells after treatment with 10 μM type C CpG-ODNs or non-CpG-ODNs. Data represent densitometric analysis of Western blots. FIG. 7D shows the effects of MMP-8 inhibitor (8 nM) and the same volume of an inactive control compound on type C CpG-ODN-induced invasion of MDA-MB-231 cells in Matrigel assays. Mean±sd, n=3. No significant differences were found between the two groups.

FIG. 8A shows a Western blot of MMP-13 (upper panel) in the various breast cancer cell lines with (+) and without (−) 10 μM type C CpG-ODN-treatment for 24 hours and after stripping of the same membrane, actin expression (lower panel) to show equal loading. FIG. 8B shows TIMP-3 expression levels from the cell lysates of the indicated breast cancer cells with or without 10 μM type C CpG-ODN-treatment for 24 hours. The 50 kDa and 21 kDa bands are from the same blot and represent different TIMP-3 forms. The same blot was stripped and reblotted with anti-actin antibody, to show equal loading of total protein. The lowest panel exhibits TIMP-3 expression in cell supernatants from similarly treated cells.

FIG. 9 shows that methylation of CpG-ODN has no effect on invasion. MDA-MB-231 cells were treated with vehicle, parental CpG-ODN or two differently methylated versions of the CpG-ODN, all at 10 μM concentration. The cells were allowed to invade for 22 h. The number of invaded cells were counted microscopically. Mean±sd, n=6 * p<0.05, *** p<0.001 vs. vehicle.

FIG. 10 shows that modification in the stem loop of CpG-ODNs affects CpG-induced invasion of MDA-MB-231 cells.

FIG. 11 shows that TLR9 agonistic CpG-ODNs induce invasion in mouse peritoneal macrophages. FIG. 11A shows PCR-verification of the mouse genotypes. For FIG. 11B, peritoneal macrophages were isolated from WT or TLR9 −/− mice and plated onto Matrigel-coated wells. The cells were treated with vehicle or with 5 μM CpG-ODNs for 22 h. The number of cells that had invaded through the membrane were counted microscopically using a 40× objective. Data represent the fold-increase in the number of invaded cells, as compared with corresponding vehicle-treated controls. Mean±sd, n=4, ** p<0.001 WT vs. TLR9 −/−.

FIG. 12 shows that CpG-induced invasion is blunted in DN TRAF6 expressing MDA-MB-231 cells. MDA-MB-231 cells were stably transfected with pIRES-EGFP empty vector (EV) or with pIRES-EGFP-TRAF6DN-Flag. Stable pools were Flow-sorted for the expression of EGFP for 3 cycles. FIG. 12A shows expression of the dominant negative TRAF6 (25 kDa) verified with Western blotting, using a TRAF6-specific antibody. The same blots were stripped and re-blotted with antibodies against Flag, GFP and actin. For FIG. 12B, the various cells were transiently transfected with a NFκB reporter gene and treated with CpG-ODN or TNF-α. Reporter gene activation was calculated as fold-increase vs. vehicle-treatment. Mean±SD, n=6. * p<0.05, *** p<0.001 pIRES-EV vs. pIRES-EGFP-TRAF6DN. For FIG. 12C, the pIRES-EGFP and pIRES-EGFP-TRAF6DN cells were plated on Matrigel-assays and treated with vehicle or with 2 or 10 μM Non-CpG-ODN or CpG-ODN, The number of invaded cells were counted microscopically 22 h later. Data are expressed as fold-increase in treatment-induced invasion, mean±sd, n±3-6. * p<0.05, ** p<0.01 pIRES-EGFP vs. pIRES-EGFP-TRAF6DN.

FIG. 13 shows intracellular TLR9 expression pattern in human MDA-MB-231 breast cancer cells.

FIG. 14 shows that TLR9 is widely expressed in human breast cancer samples. FIG. 14A shows TLR9 expression versus IgG control. FIG. 14B shows TLR9 expression in ductal breast carcinoma samples. FIG. 14C is a graph showing TLR9 staining intensity scores in normal breast epithelium, hyperplastic breast epithelium and in epithelial breast cancer cells. The mean TLR9 staining intensity scores were significantly higher in cancer cells vs. normal cells (p<0.01),

FIG. 15 shows human prostate cancer cell lines exhibit different levels of TLR9 expression. FIG. 15A shows Western blot detection of the TLR9 protein in the indicated human prostate cancer cells (upper panels). The same blots were stripped and reblotted with anti-actin antibodies, to show equal loading. FIG. 15B shows immunocytochemical detection of TLR9 in the various human prostate cancer cells. IgG was used instead of the primary antibody in the negative staining control.

FIG. 16 shows immunohistochemical detection of TLR9 in human prostate cancer tissues. A tissue array slide, containing both CaP and normal prostate samples, was subjected to immunohistochemical detection of TLR9. Each image represents a different specimen from the slide. The strongest staining for TLR9 was seen in the epithelial cancer cells from adenocarcinomas (uppermost panel). The Gleason-scores, PSA-values and clinical staging were given for each case under the immunohistochemical TLR9-staining image, Stromal staining of TLR9 was also seen in some of the adenocarcinomas (arrows). IgG was used instead of the primary antibody in the negative staining control.

FIG. 17 shows TLR9 agonistic CpG-ODNs induce invasion in TLR9 expressing prostate cancer cells in vitro. For FIG. 17A, the indicated prostate cancer cell lines were plated onto Matrigel-coated wells and treated with vehicle or with 5 μM CpG-ODNs for 20 h. The number of cells that had invaded through the membrane were counted microscopically using 40× objective, from 5 pre-destined spots. Data represent the fold-increase in the number of invaded cells, as compared, with corresponding vehicle-treated controls. Mean±sd, n−4, *** p<0,001 vs. vehicle. For FIG. 17B, cell viability of the indicated cells was measured with MTS-assays after treatment for 72 h with vehicle or with 5 μM CpG-ODNs. Mean±sd, n=6,** p<0.01, *** p<0.001 vs. vehicle. For FIG. 17C, MMP-13 activity was measured from conditioned media of PC-3 cells after treatment for 24 h with vehicle or 5 μM CpG-ODNs. Mean±sd, n=4, *** p<0.001 vs. vehicle. For FIG. 17D, PC-3 cells were treated with 5 μM CpG-ODNs, with neutralizing anti-MMP-13 antibody (12 μg/mL) or with a same amount of the corresponding control IgG. The number of invaded cells was counted as above. Mean±sd, n=4, ** p<0.01 vs. the anti-MMP-13 antibody group.

FIG. 18 shows chloroquine inhibits CpG-ODN-induced invasion in vitro. PC-3 cells were cultured for 18 h with CpG-ODN (5 μM), with a) vehicle, p38- or JNK-inhibitors (FIG. 18A), or with vehicle or chloroquine (10 μM) (FIG. 18B). The number of invaded cells was calculated as above. Data are expressed as mean±sd, n=4, ** p<0.01 vehicle vs. chloroquine.

FIG. 19 shows bacterial DNA stimulates PC-3 invasion in vitro. FIG. 19A shows PC-3 cells cultured for 20 h with CpG-ODN (5 μM) as a positive treatment-control or with indicated concentrations of E. coli DNA alone. FIG. 19B shows PC-3 cells cultured for 20 h with CpG-ODN (5 μM) as a positive treatment-control or with 1000 ng/ml E. coli DNA and chloroquine (10 μM) or the same volume of vehicle. The number of invaded cells was calculated as above. Data are expressed as mean±sd, n=4, *** p<0.01 vehicle (FIG. 19A), vehicle vs. chloroquine (FIG. 19B).

FIG. 20 shows estradiol stimulates TLR9 expression in LnCaP cells. For FIG. 20A, cells were cultured for 24 h in the presence of indicated estradiol or testosterone concentrations. The same volume of alcohol and PBS served as vehicle controls for estradiol and testosterone, respectively. TLR9 expression was studied with anti-TLR9 antibody (upper panels). The same blots were stripped and re-blotted with anti-actin antibody (lower panels), to show equal loading. For FIG. 20B, the band areas in FIG. 20A were quantified with image analysis. The columns represent the ratio of TLR9 band area/actin band area in the corresponding lanes.

### DETAILED DESCRIPTION

Provided herein are methods and compositions useful to reduce the invasiveness of a cancer cell or cells, invasiveness of cancer is associated with increased morbidity and mortality because of increased growth and metastasis of the cancer. More specifically, provided herein is a method of reducing the invasiveness of a cancer cell or cells in a subject comprising administering to the subject an effective amount of an inhibitor of Toll Like Receptor (TLR) signaling. As shown in the Examples below, direct stimulation of members of the innate immunity, such as Toll Like Receptor-9, on cancer cells stimulates their invasion. Toll-like receptors (TLRs) are evolutionarily well conserved, transmembrane proteins that are present in almost all multi-cellular organisms and that recognize patterns specific to microbial components. In mammals the TLR family is currently known to consist of 13 members, which exhibit specificity for pathogen-derived ligands. For example, TLR4 recognizes the bacterial lipopolysaccharide (LPS), whereas members of the TLR9 subfamily (TLRs 7,8,9) recognize microbial RNA and DNA. Oligonucleotides with unmethylated CpG dinucleotides mimic the immunostimulatory activity of bacterial DNA in vertebrates and are also recognized by TLR9. As used throughout, “TLR9” refers to all members of the TLR9 subfamily and all isoforms thereof.

TLRs 1,2 and 4 are expressed on the cell surface whereas TLR3 and members of the TLR9 subfamily are intracellular. More specifically, TLR9 is localized to endoplasmic reticulum, from where it is translocated to the endosomal/lysosomal compartment for ligand recognition. Upon ligand binding, the various TLRs and their associated adapters, such as MyD88 and TRIF, recruit intracellular signaling mediators which activate transcription factors, such as NF-κB. The outcome of TLR activation is an immune reaction, characterized by increased production of various pro-inflammatory cytokines and interleukins.

In humans, TLR9 is most abundantly expressed in plasmocytoid dendritic cells and in B cells whereas in mice, myeloid dendritic cells as well as macrophages and B cells also express TLR9. Interestingly, several epithelial cell types and astrocytes have also recently been reported to express various TLRs, implying that also other cells than the actual immune cells maybe important sentinels of the innate immune system. High expression of TLR9 was recently detected in clinical samples of lung cancer and in lung cancer cell lines. In these cells, stimulation of TLR9 with its agonists was shown to result in cytokine production (Droemann et al, Respir. Res. 6:1 (2005)). Responsiveness of breast cancer cells to TLR ligands and the presence of TLRs in breast milk suggest that these receptors are also expressed in breast epithelial cells.

The present application is based on the determination that a TLR mechanism enhances cancer invasiveness in TLR expressing cancer cells. More specifically, the cancer cell is a TLR9 expressing cell. Thus, provided herein is a method of reducing the invasiveness of a TLR9 expressing cancer cell or cells in a subject comprising administering to the subject an effective amount of an inhibitor of Toll Like Receptor 9 (TLR9) signaling.

The TLR inhibitors include a variety of functional nucleic acids, proteins, and small molecules. An inhibitor of TLR signaling affects TLR signaling directly by binding or blocking the receptor or indirectly by blocking a step upstream or downstream of the receptor. As used herein, the term antagonist and inhibitor are used interchangeably to include agents that either block binding of ligands to a receptor to prevent activation or that inhibit activation after the ligand has bound the receptor. One example of an inhibitor of TLR9 signaling is an inhibitor of endosomal maturation. More specifically, inhibitors of endosomal maturation useful in the methods provided herein include, but are not limited to, chloroquine, quinacrine, monesin, bafilomycin A1 and wortmannin. Other inhibitors include known antimalarials, amebicides, and antibacterial agents. Also useful in the present-methods are modified forms of chloroquine.

Proteins that inhibit TLR signaling include antibodies with antagonistic or inhibitory properties. Such antibodies are selected from antibodies that bind the receptor itself or antibodies that bind a ligand of the receptor. Similarly, the antagonistic antibody could be selected from an antibody that binds an upstream or downstream element in the signaling pathway. Also useful, in the methods described herein are inhibitors or antagonists of TLR9, MMP13, MyD88, TRAF6 and IRAK, which can be used alone or in combination with each other. The term antibodies is used herein in a broad sense and includes both polyclonal and monoclonal antibodies. In addition to intact immunoglobulin molecules, fragments, chimeras, or polymers of immunoglobulin molecules are also useful in the methods taught herein, as long as they are chosen for their ability to inhibit TRL-9 signaling. The antibodies can be tested for their desired activity using in vitro assays, or by analogous methods, after which their in vivo therapeutic or prophylactic activities are tested according to known clinical testing methods.

Monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Button et al. and U.S. Pat. No. 6,096,441 to Barbas et al.

Digestion of antibodies to produce fragments thereof, particularly, Fab fragments, can be accomplished using routine techniques known in die art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77. 1985) and by Boerner et al. (J. Immunol., 147(1):86 95,1991). Human antibodies (and fragments thereof) can also be produced using phage display libraries (Hoogenboom et al., J. Mol. Biol., 227:381, 1993; Marks et al, J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen, binding portion of an antibody. Methods for humanizing non human antibodies are well known in the art. For example, humanized antibodies can be generated according to the methods of Winter and co workers (Jones et al. Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al).

Also provided herein are functional nucleic acids that block the TLR pathways. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of TLR9.

Functional nucleic acids are nucleic acid molecules that have a specific function, such as binding a target molecule or catalyzing a specific reaction. Functional nucleic acid molecules can interact with any macromolecule, such as DNA, RNA, polypeptides, or carbohydrate chains. Thus, functional nucleic acids can interact with the mRNA, genomic DNA, or polypeptide. Often functional nucleic acids are designed to interact with other nucleic acids based on sequence homology between the target molecule and the functional nucleic acid molecule, in other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication, Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Preferred ribozymes cleave RNA or DNA substrates, and more preferably cleave RNA substrates. Representative examples of how to make and use ribozymes to catalyze a variety of different reactions can be found in U.S. Pat. Nos. 5,837,855, 5,877,022, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression, in one example, an siRNA triggers the specific degradation of homologous RNA molecules, such as mRNAs, within the region of sequence identity between both the siRNA and the target RNA, Sequence specific gene silencing can be achieved in mammalian cells using synthetic, short double-stranded RNAs that mimic tire siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands), siRNA can also be synthesized in vitro using kits such as Ambion's SILENCER® siRNA Construction Kit (Ambion, Austin, Tex.).

The compositions of the invention can be administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The disclosed compositions can be administered in a number of ways depending on whether local or systemic treatment is desired, and on the area to be treated. Thus, the disclosed compositions can be administered, for example, orally. parenterally (e.g., intravenously), by intramuscular injection, by intraperitoneal injection, transdermally, extracorporeally, or topically.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. Generally, the dosage will vary with the age, condition, sex, type of disease and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. However, an appropriate amount can be determined by one of ordinary skill in the art using only routine experimentation given the teachings herein. Chloroquine, for example, can be administered orally (for example, in tablets) at about 5-50 mg/kg of body weight daily. Chloroquine is optionally injected into a muscle (intramuscularly), under the skin (subcutaneously), or intravenously.

The materials may be in solution, suspension (for example, incorporated into microparticles, liposomes, or cells). These may be targeted to a particular cell type via antibodies, receptors, or receptor ligands. Suitable carriers and their formulations are described in Remington: The Science and Practice of Pharmacy (21th ed.) ed. David B. Troy, Lippincott Williams & Wilkins, 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody; which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agent, a chemotherapeutic agent, and the like.

Also provided herein is a method of reducing the invasiveness of a cancer cell in a subject comprising administering to the subject an effective amount of an inhibitor of Toil Like Receptor-9 (TLR9) signaling. The cancer cell can be, for example, an astrocytoma, brain cancer cell, breast cancer cell prostate cancer cell, lung cancer cell, gastric cancer cell and the like. Preferably, the cancer cell is a TLR9 expressing cancer cell. The methods described herein can also comprise the step of identifying the cancer cell as a TLR9 expressing cell.

A method of determining whether a cancer cell is capable of invasion is provided. The method comprises the step of measuring the level of expression or activity of TLR9 in the cancer cell, wherein an increase in the level of expression or activity of TLR9 as compared to control indicates that the cancer cell is capable of invasion. The level of TLR9 mRNA or protein can be measured by any assay known to those of skill in the art. For example, mRNA can be measured by densitometry using a Northen blot and protein can be measured similarly using a Western blot.

Also provided herein is a method of treating cancer in a subject, comprising the steps of (1) determining whether one or more of the subject's cancer cells are TLR (e.g., TLR9) expressing and (2) administering a CpG-motif containing: unmethylated oligonucleotides (CpG-ODNs) to the subject, if the cancer cells are negative for TLR9. CpGs optionally are used in combination therapy with an inhibitor of TLR signaling. Similarly, other agents can be used in combination with the inhibitor of TLR signaling (e.g., radiation therapy, surgery, chemotherapeutics, etc.).

A method of treating cancer in a subject is provided, comprising the steps of (1) determining whether one or more of the subject's cancer cells are TLR9 expressing cancer cells; and (2) administering a TLR9 antagonist to the subject, if the cancer cells express TLR9. As described above, the TLR9 antagonist can be an inhibitor of endosomal maturation such as, for example, chloroquine, quinacrine, monesin, bafilomycin A1 and wortmannin. The TLR9 antagonist can also be a functional nucleic acid, an antibody or a suitable small molecule.

The subjects of the provided methods can have any form of cancer such as, for example, an astrocytoma, a glioblastoma, breast cancer, prostrate cancer, brain cancer, lung cancer or gastric cancer. Preferably, however, the cancer cells of the subject express TLR9.

The methods taught herein are also useful when a subject with cancer is exposed to an infective agent known to cause cancer progression (e.g., Mycoplasma), when a subject has a history of exposure to the infective agent, or when a subject is at risk for exposure to the infective agent. Thus, provided herein is a method of reducing the deleterious effects of the infective agent by administering to the subject in need thereof an effective amount of an inhibitor of Toll Like Receptor (TLR) signaling. The method optionally further comprising determining whether a subject has been exposed to the infective agent or is at risk of exposure to the infective agent.

In additional embodiments, the inhibitor of TLR-9 signaling is administered in combination with one or more other therapeutic or prophylactic regimens, such as, for example, chemotherapy. As used throughout, a therapeutic agent is a compound or composition effective in ameliorating a pathological condition. An illustrative example of a therapeutic agent includes an anti-cancer compound, anti-inflammatory agents, anti-viral agents, anti-retroviral agents, anti-opportunistic agents, antibiotics, immunosuppressive agents, immunoglobulins, and antimalarial agents.

An anti-cancer compound or chemotherapeutic agent is a compound or composition effective in inhibiting or arresting the growth of an abnormally growing cell. Thus, such an agent may be used therapeutically to treat cancer as well as other diseases marked by abnormal cell growth. A pharmaceutical effective amount of an anti-cancer compound is an amount administered to an individual sufficient to cause inhibition or arrest of the growth of an abnormally growing cell. Illustrative examples of anti-cancer compounds include: bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine.

Inhibitors of TLR-9 signaling can be further combined with other therapies, such as chemotherapy and/or radiotherapy in the treatment of malignancy, and therapeutic efficacy can be enhanced by apoptosis-inducing compounds, such as bisindolylmaleimide VIII (BisVIII) or other sensitizing agents like SN-50 or LY294002.

Any of the aforementioned treatments can be used in any combination with the inhibitors described herein. Thus, for example, the inhibitors can be administered in combination with a chemotherapeutic agent and radiation. Other combinations can be administered as desired by those of skill in the art. Combinations may be administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

Cancer cells targeted by the methods and compositions taught herein include all TLR expressing cancer cells, and more particularly TLR9 expressing cancer cells. For example, the cancer cells are selected from the group consisting of astrocytoma, glioblastoma, breast cancer cell, lung cancer cell, and gastric cancer cell.

As used in the specification and the appended claims, the singular forms a, an and the include plural referents unless tine context clearly dictates otherwise. Thus, for example, reference to an inhibitor includes mixtures of one or more inhibitors, and the like.

Ranges may be expressed herein as from about one particular value, and/or to about another particular value. When such a range is expressed, another embodiment includes from the one particular value and/or to the other particular value. Similarly, when values are expressed as approximations, by use of the antecedent about, it will be understood that the particular value forms another embodiment. It will be further understood that the endpoints of each of the ranges are significant both in relation to the other endpoint, and independently of the other endpoint.

The terms higher, increases, elevates, or elevation refer to increases above a control. The terms low, lower, reduces, or reduction refer to any decrease below control levels. For example, control levels are in vivo levels prior to, or in the absence of, addition of an agent such as chloroquine or another inhibitor of TLR signaling. Thus, a reduction in invasiveness in the presence of an inhibitor of TLR9 signaling refers to a decrease as compared to invasiveness in the absence of the inhibitor. The reduction includes a complete elimination of the invasiveness. Inhibit, inhibiting, and inhibition mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein the terms treatment, treat or treating refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, the method for treating cancer is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

Disclosed are materials, compositions, and components that can be used for, can be used in conjunction with, can be used in preparation for, or are products of the disclosed method and compositions, These and other materials are disclosed herein, and it is understood that, when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### EXAMPLES

### Example 1

### Toll Like Receptor-9 Agonists Promote Invasion of Cancer Cells

### Materials and Methods

Chemicals. Phosphorothioate modified, human specific CpG-ODNs [type A: 5′-ggG GGA CGATCG TCg ggg gg-3′(SEQ ID NO: 1), in which only the bases that are shown in capital letters are phosphodiester, and those in lower case are phosphorothioate (nuclease resistant), type B; 5′-tcg tcg ttt tgt cgt ttt gtc gtt-3′ (SEQ ID NO:2), type C: 5′-tcg tcg tcg ttc gaa cga cgt tga t-3′ (SEQ ID NO:3)] and their non-CpG-ODN controls (type A-control: 5′-ggG GGA GCA TGC TGg ggg gc-3′ (SEQ ID NO:4), type B-control: 5′-tgc tgc ttt tgt get ttt gtg ctt-3′ (SEQ ID NO:5), type C-control: 5′-tgc tgc tgc tig caa gca get tga t-3′ (SEQ ID NO:6)) were purchased from InVivoGen (San Diego, Calif.) and dissolved into endotoxin-free sterile d-H₂O per manufacturer's suggestion and used at the indicated concentrations, Matrigels were from BD Biosciences (Bedford, Mass.), serine protease inhibitor aprotinin and MMP-inhibitor GM6001 were from EMD Biosciences (La Jolla, Calif.), MMP-8 specific inhibitor 1 and the negative control compound were from Calbiochem (San Diego, Calif.), MMP-13 immunoblotting standard (human) for the zymography was from Biomol (Plymouth Meeting, Pa.).

Cell culture. Human MDA-MB-231 breast cancer, U373 astrocytoma and D54MG glioblastoma cells were cultured in Dulbecco's modified Eagle's medium (Gibco BRL, Life Technologies, Paisley, UK) supplemented with 10% heat-inactivated fetal bovine serum, L-glutamine, penicillin/streptomycin and non-essential amino acids (all from Gibco BRL, Life Technologies). T47-D cells were cultured in RPMI, and MCF-7 cells were cultured in α-MEM, supplemented with 10% heat-inactivated fetal calf serum, 100 units/ml penicillin, 100 μg/ml streptomycin, and 2 mM glutamine and with 10 μg/ml insulin (Sigma, St. Louis, Mo.). MCF-10A cells were cultured as previously described in detail (Debnath et al., Methods 30:256-68 (2003)). All cell cultures were done in incubators in a 37° C. atmosphere of 5% CO₂/95% air

TLR mRNA expression profiling. The mRNA expression levels of the various TLRs in MDA-MB-231 cells was investigated using the SuperArray human TLR-pathway specific gene expression profiling system (SuperArray Bioscience Corp., Frederick, Md.). Briefly, total cellular RNA, was isolated using the RNAZol reagent (Tel-Test Inc., Friendswood, Tex.) from the cells grown in normal culture medium and converted to a labeled cDNA probe. The denatured cDNA was hybridized overnight at 60° C. to nylon membrane that contained the target cDNAs. Chemiluminescence was used to detect the hybridization signal on a X-ray film (Eastman Kodak Company, Rochester, N.Y.). Per manufacturer's instructions, the X-ray film was scanned with a high resolution scanner (˜300 dpi) into a JPEG-format image, converted into a TIFF-format (8-bit inverted grayscale) image by using a software Photoshop (Adobe Systems Inc. San lose, Calif.), The images were then uploaded into a software ScanAlyze (Eisen Lab, UC at Berkeley) to produce a raw intensity data sheet. The raw data from both the control and the treated groups were combined and uploaded into a software GEarrayAnalyzer (SuperArray Inc., Bethesda, Md.), where differences and ratios between the treated and the control groups were analyzed. Background was subtracted from signals and a house-keeping gene such as actin was used to calculate the ratio.

Flow-cytometry. MDA-MB-231 cells were cultured on Petri-dish (15 cmdiameter) until about 70% confluent. The cells were then detached using CellStripper (Fisher Scientific, Springfield, N.J.), and prepared for analysis using the BD Cytofix/Cytoperm Kit (BD Biosciences, San Diego, Calif.), according to the manufacturer's recommendations. Briefly, about 1×10⁶ cells were suspended into 0.5 ml of the fixative solution, After washing the cells twice, PE-conjugated anti-human TLR9 antibody (eBioscience, San Diego, Calif.) or PE-conjugated, isotype controlled IgG was added to the cells (7 μl per tube). After incubation for 30 min at 4° C., the cells were rinsed twice with PBS, and analyzed with FACS.

Immunohistochemistry. For the immunohistochemical stainings, the MDA-MB-231 cells were grown on glass coverslips in normal culture medium. The cells were fixed with 3% paraformaldehyde-PBS for 10 min at room temperature, after which they were permeabilized with 0.025% saponin for 30 min on ice. After blocking the samples with 10% goat serum, staining with mouse monoclonal antibody to TLR9 (Cat# ab 16892, Abcam Inc, Cambridge, Mass.) was performed. HRP-conjugated anti-mouse antibody was used to visualize the staining. The samples were then examined using a Leica light microscope (Wetzlar, Germany). To detect TLR9 expression in clinical breast cancer specimens, the antibody IMG-305 (Imgenex, San Diego, Calif.) was used at a dilution of 1:100.

Western blotting. The cells were cultured on 6-well plates in their normal culture medium until near confluency, after which they were rinsed with sterile PBS and cultured for further 24 h in serum-tree culture medium. The culture medium was then discarded and the cells were harvested in lysis buffer (20 mM Tris pH 7.4, 150 mM NaCl, 1 mM EDTA, 1 mM EGTA, 1% Triton, 2.5 mM sodium pyrophosphate, 1 mM β-glycerolphosphate, 1 mM Na₃ VO₄, 1 μg/ml leupeptin, (Cell Signaling, Beverly, Mass.) and clarified by centrifugation. After boiling the supernatants in reducing SDS sample buffer for 5 minutes, equal amounts of protein (˜50 μg) were loaded per lane and the samples were electrophoresed on 1.0% polyacrylamide SDS gel and transferred to a nitrocellulose membrane. TLR9 and TIMP-3 were detected with anti-TLR9 (1MG-431, Imgenex, San Diego, Calif.) and anti-human TIMP-3 (AB802, Chemicon Int., Temecula, Calif.) antibodies. MMP-13 was detected with anti-human MMP-13 antibody (R&D Systems, Minneapolis, Minn.). Binding of the primary antibodies to the target proteins on the membranes was revealed with species-specific HRP-conjugated secondary antibodies (Cell Signaling). The same blots were stripped and re-blotted, using anti-actin antibody (Sigma), to show equal loading. The protein bands were visualized by chemiluminescence using SuperSignal West Pico ECL kit (Pierce, Rockford, Ill.). Expression of TLR9 in human breast cancer specimens or in normal breast tissue was studied from specimens that were obtained from the UAB tissue repository. Briefly, the tissues were homogenized in the lysis buffer and analyzed with Western blotting as described above.

In vitro invasion assays. For the Matrigel-invasion assay the cells were plated at the density of 5×10⁴ (MDA-MB-231, U373, D54MG), 15×10⁴ (T47-D) or 30×10⁴ (MCF-7) cells per upper well in 750 μl of normal culture medium (Virtanen et al., Cancer Res. 62:2708-14 (2002)), indicated concentrations of the various CpG-ODNs, non-CpG-ODNs or vehicle were added to both the upper and lower wells. When indicated, aprotinin (2 μM), GM6001 (2 μM), MMP-8 inhibitor 1 (8 nM) or the same volume of the corresponding control compound, control IgG or neutralizing anti-MMP-13 antibody (R& D Systems, Minneapolis, Minn., both at 12 μg/ml) or chloroquine (10 μM) were also added to both upper and lower wells. The cells were allowed to invade for 18 h, after which the inserts were removed and stained with the Hema 3 Stain set (Fisher Diagnostics, Middletown, Pa.), according to the manufacturer's recommendation. The number of invaded cells was counted from 5 pre-selected microscopic fields using a 40× objective. To assess invasion in a three-dimensional type I collagen gel, acid solubilized type I collagen (0.9 ml) was added to the Costar Transwell dishes (Corning, Inc., Corning, N.Y.) and gelled over 45 min at 37° C. The collagen was prepared using rat-tail type 1 collagen dissolved in 0.2% acetic acid at 3.2 mg/ml and gelled by neutralizing the acid with 0.3 N NaOH containing phenol red as a pH indicator. A final concentration of 3.0 mg/ml was obtained. Media, containing vehicle or 10 μM type C CpG-ODN was men added to the upper and lower chambers prior to the addition of 5×10⁵ cells to the surface of the collagen gel in the presence of serum-containing medium. Media were changed every three days over the 7-day incubation period. Gels were then removed from the Transwell dish, fixed in 2.7% formaldehyde for 24 h and embedded in paraffin. Sections (6-μm) were cut and stained with hematoxylin and eosin. Tumor cell invasion (depth and number of cells below the surface) was assessed by light microscopy in a minimum of four randomly selected sections for each experimental sample. The number of invading cells per high power field (400×) were counted and averaged. The depth of invasion was also measured in four randomly selected areas for each sample using photomicrograph of each sample.

Cell viability assays. MDA-MB-231, T47-D or MCF-7 cells were plated at the density of 1000 cells/well in 96-well plates in normal culture medium, and cultured for the indicated periods of time with 10 μM type C CpG-ODN or vehicle. Cell viability was assessed after MTS was added for the final 2 hours of the experimental cultures as recommended by the manufacturer (CellTiter 96 AQueous Non-Radioactive Cell Proliferation Assay, Promega, Madison, Wis.).

Zymograms. The zymograms were performed as previously described (Suarex-Cuervo et al., Clin. Exp. Metastasis 21:525-33 (2004)). In this assay, the gelatinolytic bands represent the following MMPs: 120 kDa band represents MMP-9 and neutrophil gelatinase associated lipocalin complex, 90 kDa band represents pro-MMP-9 and the 72 kDa band represents proMMP-2. Briefly, MDA-MB-231 cells were plated on 12-well plates and allowed to reach confluency. The cells were then rinsed with PBS and serum-free medium, with the indicated concentrations of type C CpG-ODNs, type C non-CpG-ODNs or vehicle was applied for 24 h. The supernatants were then collected and a 35 μl aliquot was applied to zymograms (Novex 10% gelatin gels, Invitrogen, Carlsbad, Calif.) according to manufacturer's suggestions. In further experiments, aprotinin (2 μM) or GM6001 (2 μM) were added to the final incubations of the gels, to investigate whether CpG-treatment induced serine protease or MMP-activity.

MMP-13 ELISA, MDA-MB-231, T47-D and MCF-7 cells were plated on 24-well plates at the density of 10⁵ cells per well and allowed to reach confluency. The cells were then rinsed with PBS and 200 μl of serum-free medium, containing vehicle or 10 μM type C CpG-ODNs were added per wells. The supernatants were collected 24 h later and analyzed for levels of active MMP-13 with an ELISA that detects active MMP-13 (Calbiochem, La Jolla, Calif.), according to the manufacturer's instructions.

MMP-8 analysis. The molecular forms and degree of activation of MMP-8 were analyzed by Western immunoblotting, using anti-rabbit MMP-8 antibody, After SDS-Page run under nonreducing conditions, the proteins on the gel were transferred onto a nitrocellulose filter (Bio-Rad Laboratories, Richmont, Calif.). After blocking with 3% gelatin, the membrane first reacted with the primary antibody (1:500) and then with alkaline phosphatase conjugated secondary antibody. Immunoreactive proteins were visualized by nitro blue tetrazolium (Sigma) and 5-bromo-4-chloro-3-indolyl-phosphate (Sigma). Quantitation was done with the Bio-Rad Model Gs-700 Imaging Densitometer, using the Analyst program. Data are expressed as densitometric arbitrary units. Human neutrophil and rheumatoid synovial culture media were used as positive controls for PMN-type and mesenchymal type MMP-8 isoforms, respectively.

Statistical analysis. The results are given as mean±sd, unless otherwise stated. Student's t test was used to calculate statistically significant differences between the various study groups.

Results

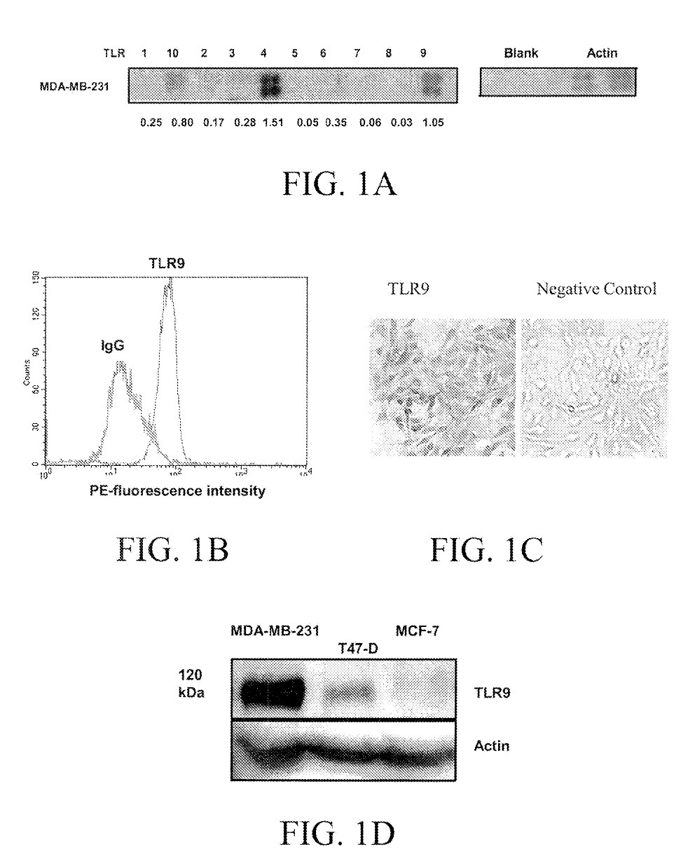

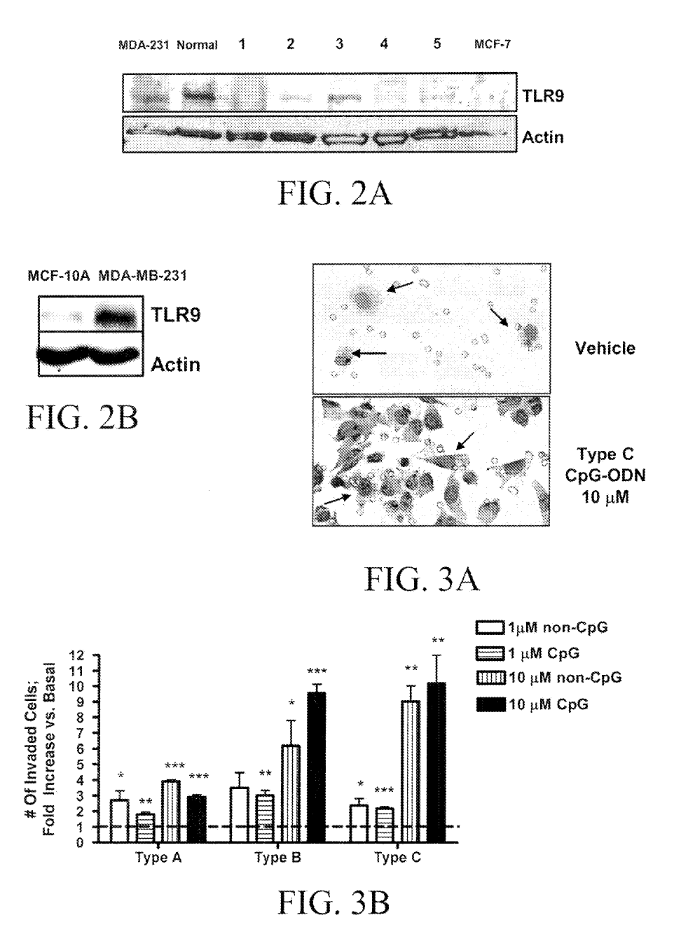

TLR9 is expressed in breast cancer cell lines and in clinical samples of breast cancer. MDA-MB-231 cells express relatively high levels of mRNAs for TLR4 and TLR9, but only very little or no mRNAs for the other TLRs 1-10, as detected in DNA-arrays (FIG. 1A). The present study focused on TLR9 expression and function. Flow cytometry and also immunohistochemistry of the permeabilized MDA-MB-231 cells suggested intracellular expression of TLR9, as also shown previously in other cells (FIGS. 1B and 1C) (Wagner, Trends Immunol 25:381-6 (2004); Latz et al., Nat. Immunol 5:190-8 (2004)). Anti-TLR9 antibody detected a high level of expression of a band about 120 kDa in MDA-MB-231 cells and an intermediate expression level in T47-D cells, but no specific signal was seen in MCF-7 cells in Western blots (FIG. 1D,), TLR9 expression was also detected with Western blot in normal mammary gland tissue and in 3 out of 5 malignant breast tumors. Interestingly, the TLR9 band detected in the normal mammary gland tissue appeared slightly heavier than the TLR9 band in the malignant tumors and in the MDA-MB-231 cells (FIG. 2A). The same blot was stripped and re-blotted with anti-CD45 antibody, which is a pan-leukocyte marker. As no specific expression of CD45 was seen, the TLR9 expression in these lysates was from the epithelial cells of the breast. TLR9 expression was also detected in immortalized human breast epithelial cell line MCF-10A (FIG. 2B), Taken together, these results show that TLR9 is expressed in both normal and cancerous mammary epithelial cells.

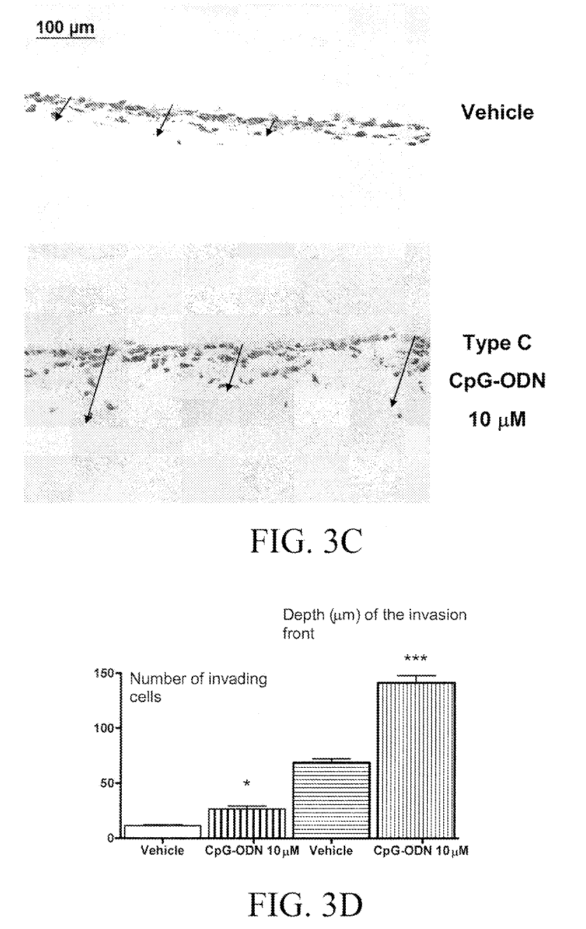

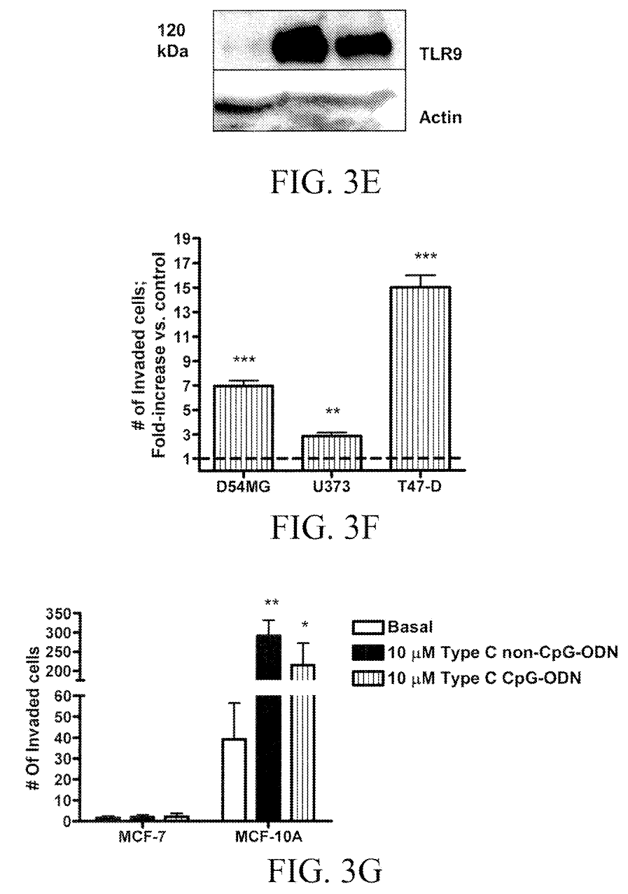

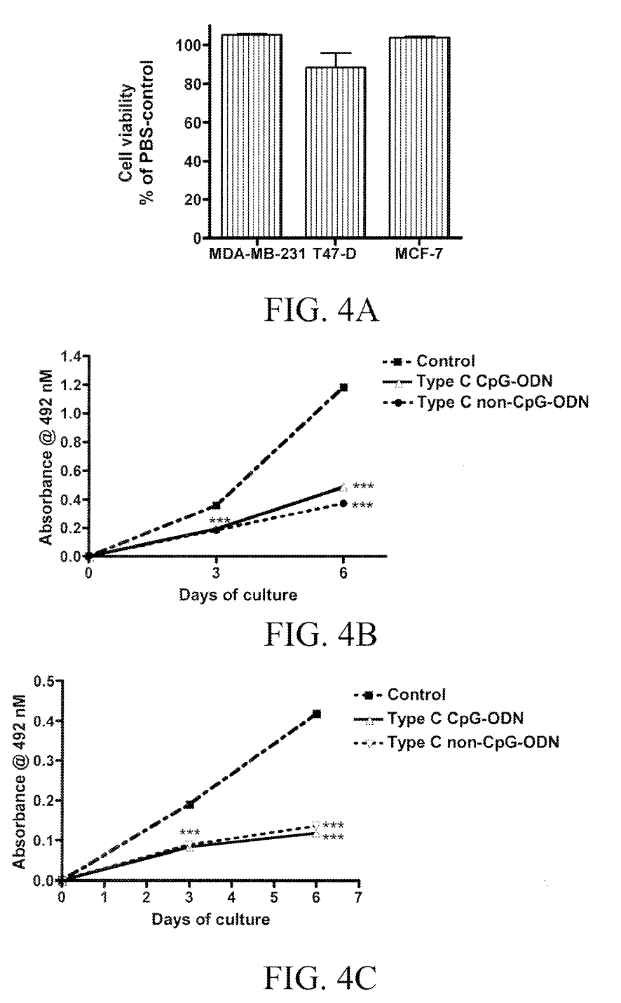

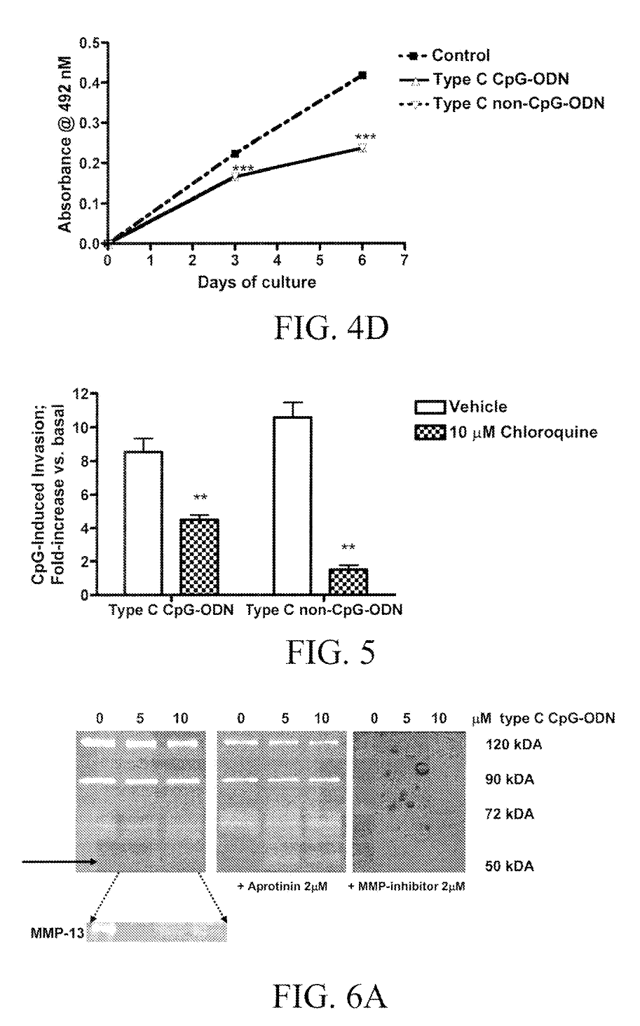

TLR9 agonists induce invasion in TLR9 expressing cancer cells. To study the effects of TLR9 stimulation on breast cancer behavior, cell invasion and cell viability assays were performed using the well-characterized TLR9 agonists, CpG-motif containing unmethylated oligonucleotides (CpG-ODN), which mimic the actions of bacterial DNA. Three different CpG-ODNs (types A, B and C), with slight variations in their sequences, were tested. Types B and C represent CpG-ODNs with the conventional nuclease-resistant phosphorothioate-backbone and type A carries a combination of phosphorothioate- and phosphodiester modifications. Such modifications resulted in a slightly different cytokine response from dendritic cells at 0.01-10 μM concentrations (Hemmi et al, J. Immunol. 170:3059-64 (2003)). All these CpG-ODNs induced a dose-dependent increase in the number of MDA-MB-231cells that invaded through Matrigel. The treatment-induced increased invasion ranged from 2- to 10-fold and was statistically significant even with the lowest, 1 μM concentrations tested. Surprisingly, also the non-CpG-ODNs, which are considered unstimulatory controls for the TLR9-agonistic CpG-ODNs, induced invasion of MDA-MB-231 cells to a similar level. The type C CpG-ODN was chosen for further studies, since if is a combination of types A and B CpG-ODNs and since it induced, along with the type B CpG-ODNs, the highest dose-responsiveness (FIGS. 3A and 3B), Similar effects on invasion were also seen when MDA-MB-231 cells were cultured for 7 days in the presence of 10 μM type C CpG-ODNs in 3-dimensional collagen culture assays (FIGS. 3C and 3D), The type C CpG-ODNs (10 μM) stimulated invasion also in the unrelated, strongly TLR9-expressing U373 astocytoma and D54MG glioblastoma cells and in T47-D breast cancer cells (FIGS. 3E and 3F). Treatment with the type C CpG-ODNs or with the type C non-CpG-ODNs (1 and 10 μM) stimulated invasion also in the immortalized mammary epithelial cell line MCF-10A, but they did not, however, stimulate invasion in the TLR9 negative MCF-7 breast cancer cells (FIGS. 3E and 3G). The increased ceil numbers seen in the invasion assays were not due to an effect on proliferation or apoptosis, because the type C CpG-ODN (10 μM) had no effect on cell viability during an incubation of 24 h (FIG. 4A). During longer incubation, both type C non-CpG-ODN and type C CpG-ODN actually significantly decreased viability, as detected with MTS-assays (FIGS. 4B, 4C and 4D). Taken together, these studies showed that TLR9 agonists stimulate invasion in TLR9+ but not in TLR9- cancer cells. To further study the role of TLR9 in mediating invasion, the invasion assays were performed with and without chloroquine, an inhibitor of endosomal maturation, which has been shown to prevent TLR9 signaling in other cells (Rutz et al, Eur. J. Immunol. 34:2541-50 (2004)). As shown in FIG. 5, addition of chloroquine (10 μM) inhibited both CpG-ODN- and non-CpG-ODN-induced invasion. Similar results were obtained also with higher chloroquine doses (25 μM).

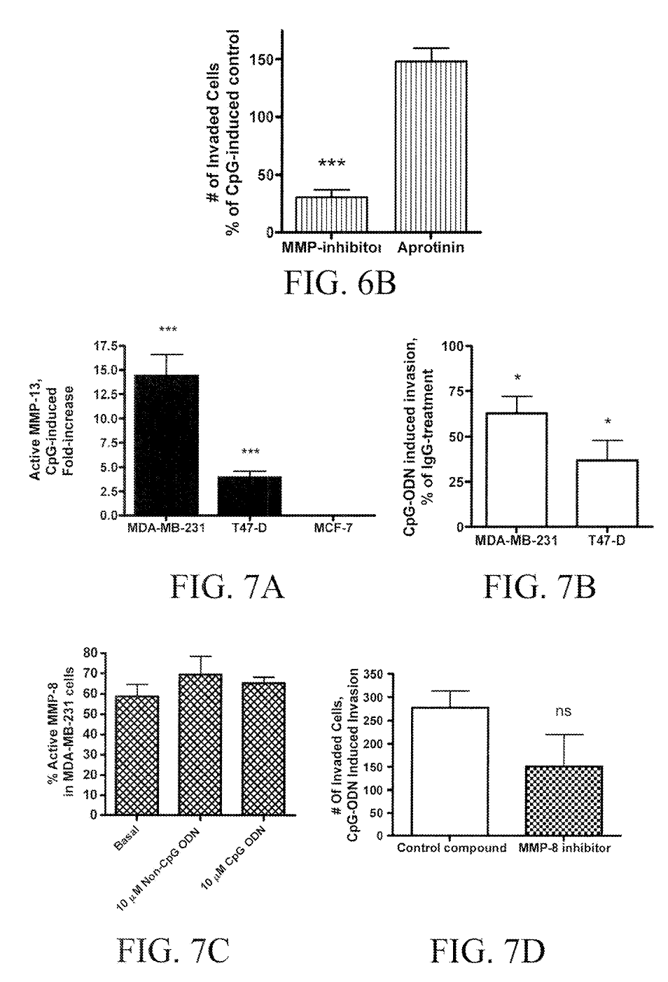

TLR9 agonists induce matrix metalloproteinase activity. To investigate the mechanism behind the TLR9-agonist-induced invasion, gelatin-zymogram assays were performed. Supernatants from MDA-MB-231 cells were treated with 5 or 10 μM type C CpG-ODNs or for 24 h induced the formation of a gelatinolytic band of about 50 kDa. The appearance of this band was not inhibited with the serine protease inhibitor aprotinin, but it did disappear when the gels were incubated with the broad spectrum matrix metalloproteinase inhibitor GM6001, The size of the band was similar to that induced by a positive MMP-13 control sample. (FIG. 6A), Similar results were obtained with the type C non-CpG-ODN. Consistent with these findings, the type C CpG-induced invasion of MDA-MB-231 cells was also inhibited by the MMP inhibitor, but not by the serine protease inhibitor aprotinin in Matrigel-assays (FIG. 6B),

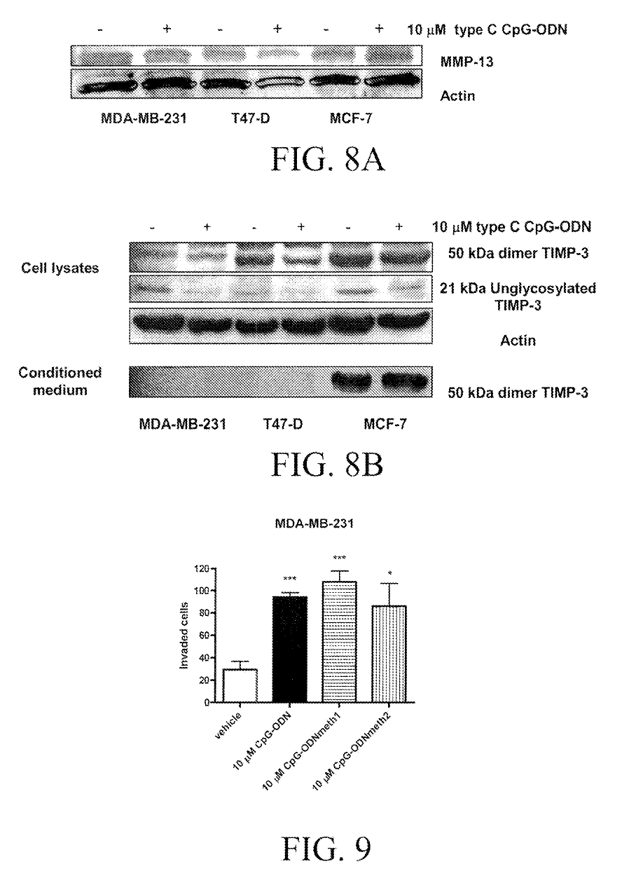

TLR9 agonist-induced invasion can be blocked with neutralizing anti-MMP-13 antibodies. Based on the molecular weight of the CpG-ODN-induced gelatinolytic band, it was hypothesized that this treatment induced the activation of MMP-13. ELISA analysis of the supernatants of the MDA-MB-231 breast cancer cells treated either with vehicle or with 10 μM type C CpG-ODNs indeed revealed significantly increased levels of active MMP-13 in the TLR9-agonist treated supernatants, as compared with those of the vehicle treated cells. Similar induction was seen in the TLR9+ T47-D cells, but not in the TLR9- MCF-7 cells (FIG. 7A). Neutralizing antibodies to MMP-13 also blocked the type C CpG-ODN induced invasion of MDA-MB-231 and T47-D cells, whereas control antibodies did not (FIG. 7B). Treatment with type C CpG-ODN (10 μM) did not, however, affect the expression of total or active MMP-8 in these cells, as judged by Western immunoblotting (FIG. 7C). Specific inhibitor of MMP-8 inhibited basal invasion (#of invaded cells, mean±sd, 50±9 vs 17±3, basal vs. MMP-8 inhibitor, respectively, p<0.05), but it did not block CpG-ODN-induced invasion (FIG. 7D). Taken together, these findings showed that CpG-ODN (and non-CpG-ODN)-induced invasion is mediated via MMP-13. Type C CpG-ODN treatment did not, however, increase MMP-13 expression, suggesting that these TLR9 agonists stimulate breast cancer invasion via activating MMP-13 (FIG. 8A). The TLR9 agonist did not affect the expression of PAI-1, PAI-2 or TIMP-1, which can regulate uPA- and MMP-mediated invasion. Differences in the expression levels of TIMP-3, which can inhibit MMP-13 activity, were detected. Treatment with the type C CpG-ODN decreased the expression of TIMP-3 in the cell lysates of TLR9+ MDA-MB-231 and T47-D cells, but not in the TLR9-MCF-7 cells. (FIG. 8B). High levels of TIMP-3, which were not affected by the CpG-ODN-treatment, were detected only in the supernatants of the MCF-7 cells.

### Example 2

### CpG-ODN-Induced Invasion of Cancer Cells

Materials and Methods

Chemicals. Phosphorothioate modified, mouse and human specific CpG-ODNs were purchased from InVivoGen (San Diego, Calif.). Sequences and the methylation sites of the modified CpGs are given in Table 1.

TABLE 1

Sequences and Methylation Sites of Modified CpGs.
Sequences 5′ to 3′

| | |
|---|---|
| Parental | tcg tcg tcg ttc gaa cga cgt tga t |
| CpG-ODN | (SEQ ID NO:8) |
| Meth 1 | tcg tcg tcg ttc gaa cga cgt tga t |
| | (SEQ ID NO:9) |
| Meth 2 | tcg tcg tcg ttc gaa cga cgt tga t |
| | (SEQ ID NO:10) |

Nucleotides in bold font are methylated.

All cytosines in Meth 1 (SEQ ID NO:9) are methylated. Only the cytosine in the stem loop of Meth 2 (SEQ ID NO:10) is methylated.

All ODNs were dissolved into endotoxin-free, sterile d-H₂O per manufacturer's suggestion and used at the indicated concentrations. Matrigels were from BD Biosciences (Bedford, Mass.).

Cell culture. MDA-MB-231 cells were cultured in Dulbecco's modified Eagle's medium (Gibco BRL, Life Technologies, Paisley, UK) supplemented with 10% heat-inactivated fetal bovine serum, L-glutamine, penicillin/streptomycin and non-essential amino acids (all from Gibco BRL, Life Technologies). All cell cultures were done in incubators in a 37° C. atmosphere of 5% CO₂/95% air.

Stable transfection of TRAF6 into MDA-MB-231 breast cancer cells. The cDNA encoding for dominant negative TRAF6 has been described previously (Yeo et al., (2003) J. Biol. Chem. 278:22563-22573). The pIRES-EGF-DNTRAF6-Flag and pIRES-EGF were stably transfected into the MDA-MB-231 cells. The established stable pools were submitted to three cycles of Flow-sorting for highest emission of GFP, to enrich the populations of transfected cells. Stable expression of the transfected genes were confirmed in the cell lysates with Western blotting, using anti-Flag (Sigma, St. Louis, Mo.), anti-GFP (Sigma, St. Louis, Mo.), and anti-TRAF6 (Santa Cruz Biotechnology, Santa Cruz, Calif.) antibodies.

Macrophage isolation. The homozygous TLR9 knockout (TLR9 −/−) mice were made in the C57/B6 background and have been previously described in detail (Hemmi et al., Nature 408:740-5 (2000)). These mice were purchased from (Bioindustry Division Oriental Yeast Co., Ltd. Azusawa, Tokyo, Japan), wild-type control C57/B6 mice were purchased from Harlan (Indianapolis, Ind.). The mouse genotypes were confirmed by PCR-analyses run from tail snip DNA, according to the PCR protocol provided by the vendor. All procedures were performed according to the institutional animal care and use committee guidelines. Peritoneal macrophages were isolated by injecting 5-10 ml ice-cold, sterile PBS into the peritoneal cavities of the mice immediately after sacrifice. The abdomen was gently massaged and PBS then aspirated into the same syringe. The obtained cells were pooled, spun down at 800 rpm for 8 min at RT and re-suspended into normal culture medium (DMEM, 10% FCS, as above). For splenic macrophages, the mouse spleens were aseptically removed from the WT or TLR9 −/− mice and placed in a tissue cell culture dish containing 5 ml normal culture medium in the laminar hood. Several cuts were made with a scalpel on the spleen, which was men gently rubbed with a syringe piston to induce release of cells into the medium. Cells were collected by centrifugation (8 min, 800 rpm) and the cell pellet was resuspended in 10 ml of DMEM containing 10 % FCS, penicillin-streptomycin and 10 ng/ml M-CSF (R&D Systems, Minneapolis, Minn.).

In vitro invasion assays. For the Matrigel-invasion assays, the indicated cells were plated at the density of 5×10⁴ (parental and transfected MDA-MB-231 cells) or at 1×10⁴ macrophages per upper well in 500 μl of normal culture medium. Indicated concentrations of the various CpG-ODNs, were added to both the upper and lower wells. The cells were allowed to invade for 20 h (MDA-MB-231 cells and transfected pools) or for 48 h (macrophages), after which the inserts were removed and stained with the Hema 3 Stain set (Fisher Diagnostics, Middletown, Pa.), according to the manufacturer's recommendation. The number of invaded cells were counted from 5 pre-selected microscopic fields using a 40× objective.

Zymograms and MMP-13 ELISA. The cells were plated on 24-well plates at the density of 10⁵ cells per well and allowed to reach confluency. The cells were then rinsed with PBS and 150 μl of serum-free medium, containing vehicle or 5 μM type C CpG-ODNs was added per wells. The supernatants were collected 24 h later and analyzed for levels of active MMP-13 with an ELISA that detects active MMP-13 (Calbiochem, La Jolla, Calif.), according to the manufacturer's instructions.

TLR9 immunohistochemistry. Patient samples were obtained upon diagnosis from breast cancer patients that were being treated for their condition at the University Hospital of Oulu, Finland. Sections of paraffin embedded blocks were cut with a microtome and were routinely dewaxed. The specimens were subjected to immunohistochemical detection of TLR9 using IMG-305 antibody (Imgenex, San Diego, Calif.) at a dilution of 1:100.

Statistical analysis. The results are given as mean±sd. Student's t test was used to calculate statistically significant differences between the various study groups.

### Results

Methylation does not affect CpG-ODN induced invasion in breast cancer cells. As shown herein, synthetic ligands of TLR9 induce invasion in human MDA-MB-231 breast cancer cells. TLR9 mediated effects are methylation dependent and independent. To investigate whether methylation affects the invasion-inducing capacity of CpG-ODN, the central cytosines or all cytosines in this oligonucleotide were methylated, Such modified molecules were then added to MDA-MB-231 cells in the invasion assays in vitro. All CpG-ODNs induced invasion to a similar level (FIG. 9). Therefore, methylation does not affect the invasion-inducing capacity of these TLR9 ligands

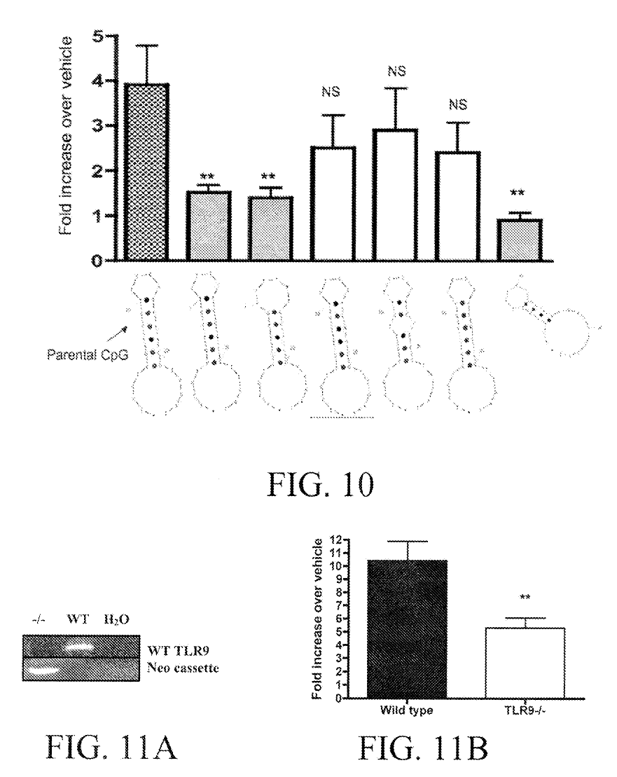

Invasion can be altered by modifying the stem-loop structure. Secondary and tertiary structures of the TLR9 ligands have been shown to be important determinants of their activity in inducing inflammatory responses. It was determined how modifications of the CpG-ODN stem loop structure affects their invasion-inducing capacity. Modifications of the loop blunted the invasion-inducing effect, as compared with the parent CpG-ODN molecule. On the contrary, modification of the stem had no effect. (FIG. 10).

CpG-ODN-induced invasion is blunted in TLR9 −/− macrophages. To further characterize the role of TLR9 in this process, macrophages that were isolated from the peritoneal cavities of TLR9 −/− and WT mice were treated. The genotypes of the mice were first verified with PCR (FIG. 11A). The cells were then plated and their invasion in response to the TLR9-agonistic CpG-ODNs were studied. All treatments induced a significant increase in cellular invasion, as compared with vehicle treatment. In the TLR9 −/− cells this effect was, however, blunted as compared with the WT mice. (FIG. 11B).

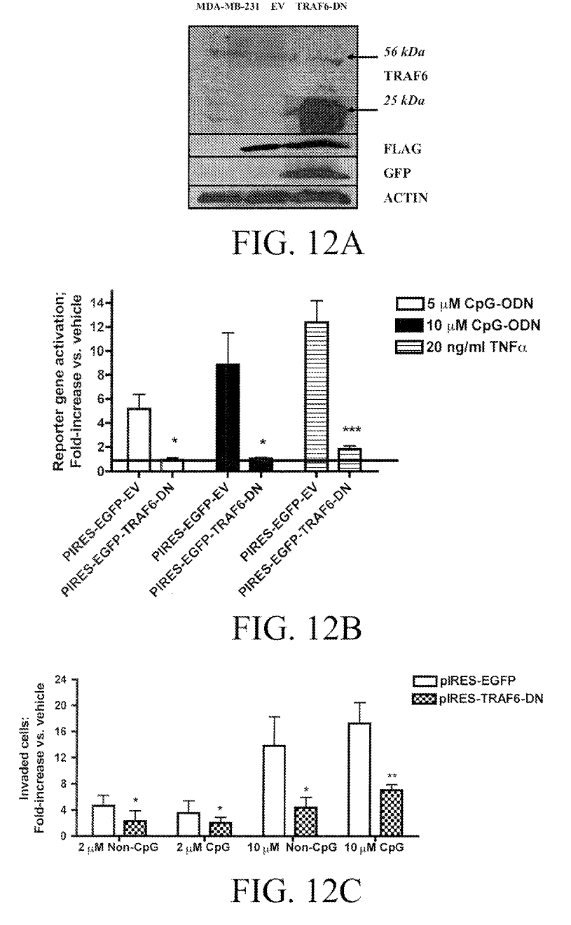

Expression of dominant negative TRAF6 blunts CpG-induced invasion. TRAP6 is a down-stream signaling mediator of TLR9. To study the role of TRAF6 in CpG-ODN-induced invasion, human MDA-MB-231 breast cancer cells were stably transfected with a dominant negative form of TRAF6 that is missing the amino-terminal end of the protein. After FACS-sorting for EGFP-expression with Flow-cytometry for three cycles, specific protein expression was investigated in the various pools. As expected, GFP-expression was detected only in the pools that were stably transfected with the pIRES-vector. Expression of Flag was detected only in the pool that was transfected with the pIRES-TRAF6DM-Flag fusion protein producing vector. Finally, a band of ˜25 kDa expected size of the truncated TRAF6 protein was only detected in the pool of cells. For comparison, all cells expressed the ˜56 kDa endogenous TRAF6 protein (FIG. 12A). The function of the DN TRAF6 was verified with NFκβ-reporter gene assays. In empty vector cells, treatment with CpG-ODNs or with TNF-α induced a significant increase in NFκβ-promoter activity. In the TRAF6DN-pool, these effects were blunted suggesting that the transfected TRAF6DN gene functions in these cells in a dominant, negative fashion (FIG. 12B). The effect of CpG-ODN-treatment on the invasive characteristics in these pools was then tested. CpG-ODNs induced a significant induction in invasion of the empty vector transfected pools. In the pools over-expressing DN TRAF6, the effect was blunted (FIG. 12C).

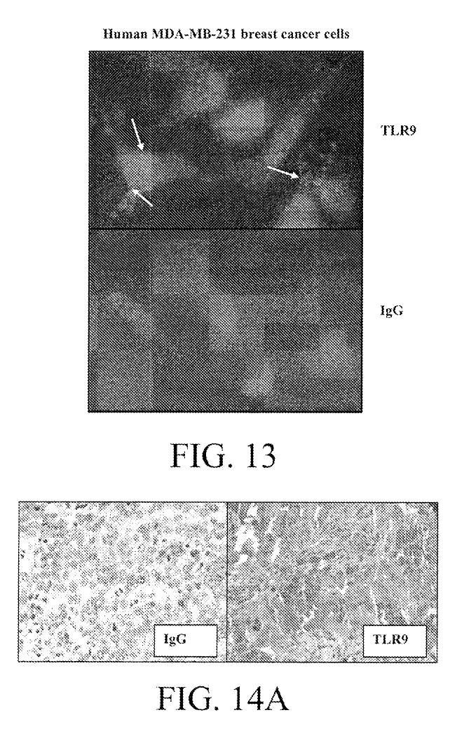

The intracellular staining pattern of TLR9 in human breast cancer cells was characterized. Human MDA-MB-231 breast cancer cells exhibit punctate, intracellular TLR9 staining (FIG. 13).

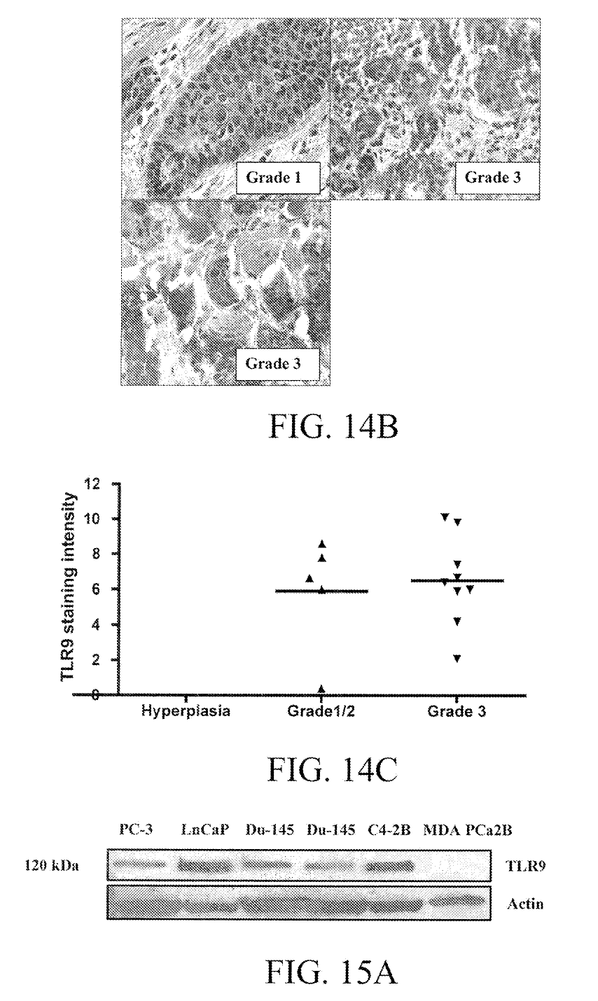

TLR9 is widely expressed in clinical breast cancer samples. TLR9 is expressed in protein lysates of clinical breast cancer samples. To verify the cellular localization of TLR9 in breast cancer, immunohistochemical staining for TLR9 was performed. Most samples (95%) exhibited TLR9 staining (FIGS. 14A and 14B). Interestingly, TLR9 staining could be detected only in the epithelial breast cancer cells and not in the stromal cells. TLR9 staining scores were significantly higher in breast cancer epithelial cells as compared to normal epithelial cells (p<0.01) (FIG. 14C).

### Example 3

### TLR-9 Agonists Stimulate Prostate Cancer Invasion

Materials and Methods

Chemicals. Phosphorothioate modified, human specific CpG-ODNs (type C: 5′-tcg tcg tcg ttc gaa cga cgt tga t-3′) (SEQ ID NQ:7) were purchased from InVivoGen (San Diego, Calif.) and dissolved into endotoxin-free sterile d-H₂O per manufacturer's suggestion and used at the indicated concentrations. Matrigels were from BD Biosciences (Bedford, Mass.). The synthetic inhibitors of p3S (SB203580), JNK (cell permeable JNK Inhibitor I, L-form) were obtained from Calbiochem (San Diego, Calif.). β-estradiol was from Sigma (St. Louis, Mo.) and testosterone enanthate (Delatestryl, BTG Pharmaceutical Corp., Iselin, N.J.) from local pharmacy.

Cell culture. Human PC-3, LnCaP, MDA PCa2b and Du-145 prostate cancer cells were originally obtained from ATCC (Manassas, Va.). PC-3, LnCap and Du-145 cells were cultured in Dulbecco's modified Eagle's medium (Gibco BRL, Life Technologies, Paisley, UK) supplemented with 10% heat-inactivated fetal bovine serum, L-glutamine, penicillin/streptomycin and non-essential amino acids (all from Gibco BRL, Life Technologies), MDA PCa2b cells were cultured as previously described in detail (Kara et al., Cancer Res. 63:437-47 (2006)). All cell cultures were done in incubators in a 37° C. atmosphere of 5% CO₂/95% air.

Western blotting. The cells were cultured in 6-well plates in their normal culture medium, until near confluency, after which they were rinsed with sterile PBS and cultured for further 24 h in serum-free culture medium. When the effects of testosterone and estradiol were investigated, the cells were cultured in the presence of charcoal-stripped serum for 48 h after plating before the actual treatment. The culture medium was then discarded and the cells were harvested in lysis buffer (20 mM Tris pH 7.4, 150 mM NaCl, 1 mM EDTA, 1 mM EGTA, 1% Triton, 2.5 mM sodium pyrophosphate, 1 mM β-glycerolphosphate, 1 mM sodium orthovanadate (Na₃VO₄), 1 μg/ml leupeptin, Cell Signaling, Beverly, Mass.) and clarified by centrifugation. After boiling the supernatants in reducing SDS sample buffer for 5 minutes, equal amounts of protein (˜50 μg) were loaded per lane and the samples were electrophoresed on 10% polyacrylamide SDS gel and transferred to a nitrocellulose membrane. TLR9 was detected with anti-TLR9 (IMG-431, Imgenex, San Diego, Calif.) antibody. The same blots were stripped and re-blotted, using anti-actin antibody (Sigma, St. Louis, Mo.), to show equal loading. Binding of the primary antibodies to the target proteins on the membranes was revealed with species-specific HRP-conjugated secondary antibodies (Pierce, Rockford, Ill.). The protein bands were visualized by chemiluminescence using SuperSignal West Pico ECL kit (Pierce, Rockford, Ill.). When indicated, the Western blot band area analysis was performed with the ImageJ image analysis software version 1.36b (NIH, U.S.A.).

Immunohistochemistry. For the immunohistochemical stainings, the cells were grown on glass coverslips in normal culture medium. The cells were fixed with 3% paraformaldehyde-PBS for 10 min at room temperature, after which they were permeabilized with 0.025% saponin for 30 min on ice. After blocking the samples with 10% goat serum, staining with mouse monoclonal antibody to TLR9 (Cat#ab 16892, Abeam Inc. Cambridge, Mass.) was performed by applying the diluted antibody 1:20 in. TBS to the coverslips. HRP-conjugated anti-mouse antibody diluted 1:500 in TBS was used to visualize the staining. The samples were then examined using a Zeiss light microscope (Thornwood, N.Y.). Expression of TLR9 in clinical PCa specimens was studied with the Imgenex human prostate cancer array #IMH-303(Imgenex, San Diego, Calif.). This array contains representative areas of ail the 40 adenocarcinoma and 9 normal prostate samples. For each cancer specimen, information about the Gleason's score, clinical staging and pre-surgery serum PSA (ng/ml) values are given by the vendor.

In vitro invasion assays. For the Matrigel-invasion assay the cells were plated at the density of 5×10⁴ (PC-3, Du-145, LnCaP and MDA PCa 2b) cells per upper well in 500 μl of normal culture medium, Indicated concentrations of the various CpG-ODNs, bacterial DNA (E. Coli, Sigma, St. Louis, Mo.) or vehicle were added to both the upper and lower wells. When indicated, p38- or JNK-inhibitor (both at final concentration of 10⁻⁵ M), control IgG or neutralizing anti-MMP-13 antibody (R&. D Systems, Minneapolis, Minn., both at 12 μg/ml) or chloroquine (10 μM) were also added to both upper and lower wells. The cells were allowed to invade for 20 h, after which the inserts were removed and stained with the Hema 3 Stain set (Fisher Diagnostics, Middletown, Pa.), according to the manufacturer's recommendation. The number of invaded cells were counted from 5 pre-selected microscopic fields using a 40× objective.

In vitro growth assays. The cells were plated on 96-well plates in normal culture medium (10³ cells in 100 μl of medium per well) and treated for 72 hours with 5 μM CpG-ODNs. To measure cell viability, MTS-assays (Cell Titer 96 Aqueous One, Promega, Madison, Wis.) were used according to the manufacturer's instructions.

MMP-13 ELISA. The cells were plated on 24-well plates at the density of 10⁵ cells per well and allowed to reach confluency. The cells were then rinsed with PBS and 150 μl of serum-free medium, containing vehicle or 5 μM type C CpG-ODNs was added per wells. The supernatants were collected 24 h later and analyzed for levels of active MMP-13 with an ELISA that detects active MMP-13 (Calbiochem, La Jolla, Calif.), according to the manufacturer's instructions.

Statistical analysis. The results are given as mean±sd. Student's t test was used to calculate statistically significant differences between the various study groups.

Results

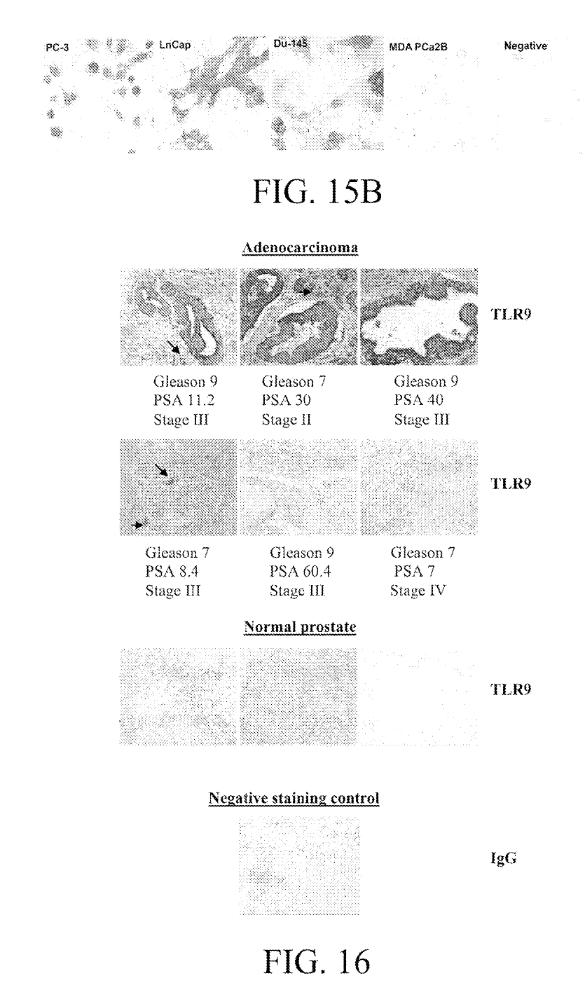

TLR9 is expressed hi human prostate cancer cell lines and in clinical samples of prostate cancer. Human prostate cancer cell lines exhibit various expression levels of the ˜120 kDa TLR9 protein, as detected with Western blotting. High TLR9 expression levels were detected in the LnCaP and C4-2B cells, intermediate levels in Du-145 and PC-3 and no expression of TLR9 was detected in the MDA Pca2b prostate cancer cells (FIG. 15A). The results were confirmed with immunocytochemistry (FIG. 15B). TLR9 immunohistochemistry was performed on a tissue array containing 40 samples of CaP adenocarcinoma and 9 samples of normal prostate tissue. Of all the samples viewed, three adenocarcinoma specimens (# 1,2 and 3, referring to the sample order on the tissue array slide) exhibited the highest staining intensity. In these samples, remarkably high TLR9 staining was detected especially in the epithelial cancer cells although some irregular staining was also seen among the stromal cells. In some PCa samples (#11) only stromal TLR9 staining, without expression in the epithelial cells was seen. Among the reviewed adenocarcinoma samples (n=40), there was no association between Gleason's score, clinical staging or serum PSA-concentrations with TLR9-staining intensity. In the remaining PCa specimens, TLR9 expression was low and similar to that in normal prostate which was taint and not localized in the epithelial cells (FIG. 16). Taken together, these results indicate that TLR9 is expressed in human prostate cancer,

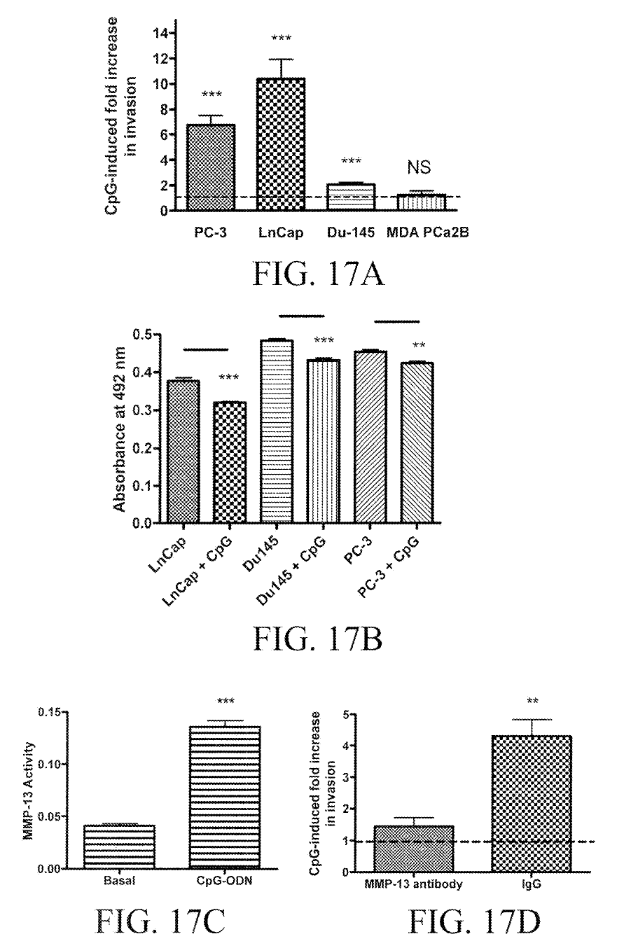

TLR9 agonists induce invasion in TLR9 expressing prostate cancer cells via induction of MMP-13. As shown herein, stimulation of TLR9 with their agonistic CpG-containing ligands results in MMP-13 mediated, increased invasion in TLR9* human breast and brain cancer cells. To study the effects of TLR9 stimulation on the invasive behavior of prostate cancer cells, invasion assays were performed using the well-characterized TLR9 agonists, CpG-motif containing unmethylated oligonucleotides (CpG-ODN), which mimic the actions of bacterial DNA in inducing an inflammatory reaction. TLR9-agonistic CpG-ODNs induced a 2-11-fold increase in all the studied TLR9⁺ prostate cancer cells, but not in the TLR9⁻ MDA Pca2b cells (FIG. 17A). This result was not affected by proliferative effects of these cells because the CpG-ODN treatment decreased the viability of these cells when cultured for 72 hours (FIG. 17B). To investigate whether CpG-ODNs increase prostate cancer cell invasion also via increased MMP-13 activation, the cells were stimulated with CpG-ligands for 24 h and measured activated MMP-13 in the conditioned supernatants with ELISA. As shown in FIG. 17C, treatment with CpG-ODNs significantly induced MMP-13 activity in the TLR9⁺ PC-3 cells. No such increase in MMP-13 activity was detected in the TLR9⁻ MDA Pca2b cells, where MMP-13activity remained below detection limit, both, before and after CpG-ODN treatment. Addition of neutralizing anti-MMP-13 antibodies to the assays inhibited significantly CpG-ODN-induced invasion of PC-3 cells, whereas addition of control IgG antibody did not (FIG. 17D). Taken together, the results indicate that CpG-ODN-induced invasion is mediated via induction of MMP-13 also in human prostate cancer cells.

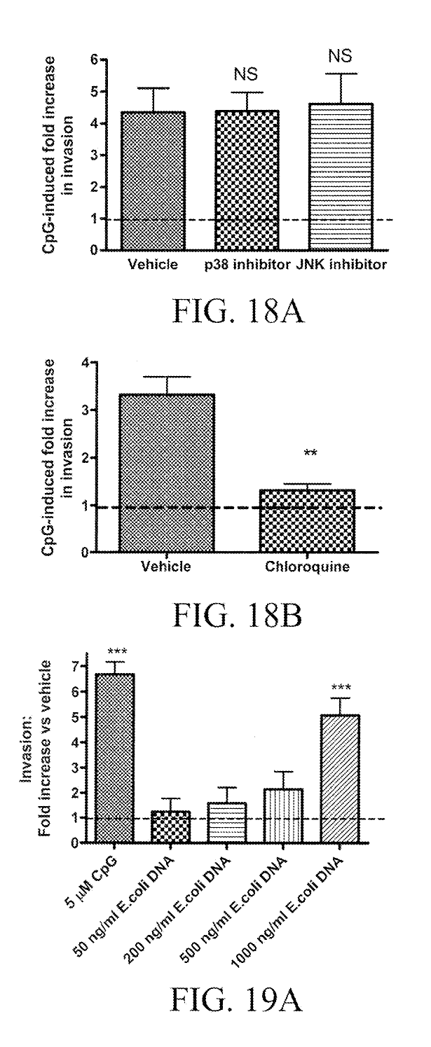

Chloroquine inhibits CpG-ODN-induced invasion of TLR9⁺ prostate cancer cells. The involvement of various downstream-signaling molecules was investigated in the CpG-ODN-induced invasion. For example, both p38 and JNK have been shown to mediate TLR9-mediated inflammatory responses. To investigate if these MAP-kinases mediate also CpG-ODN-induced invasion, the TLR9⁺ PC-3 cells were treated with CpG-ODNs, with or without synthetic inhibitors of p38 or JNK and investigated the effects on invasion using Matrigel-assays. Neither of these inhibitors affected CpG-ODN-induced invasion (FIG. 18A). Chloroquine is an inhibitor of endosomal acidification, resulting in inhibition of TLR9-signaling (Macfarlane and Manzel, J. Immunol. 160:1122-31 (1998)). As shown in FIG. 18B, chloroquine significantly inhibited CpG-ODN-induced invasion in PC-3 cells. Taken together, these results indicate that the CpG-ODN-induced invasion is TLR9-mediated.

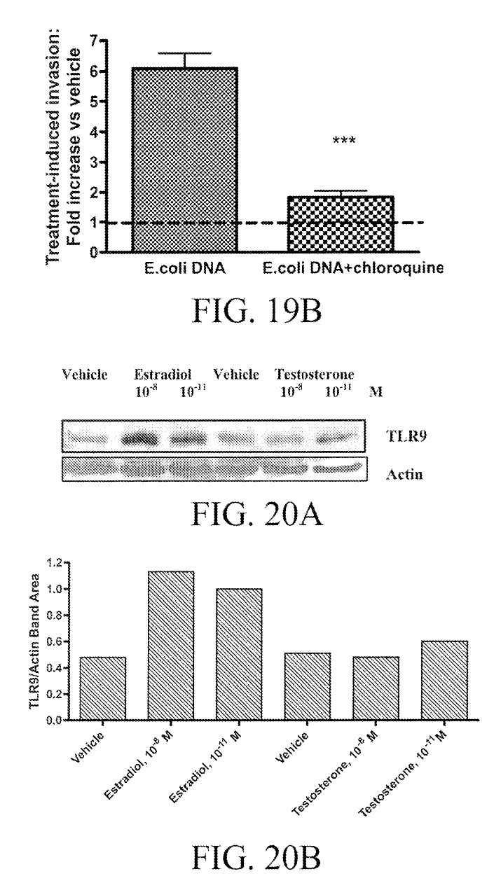

Bacterial DNA induces invasion in TLR9⁺ prostate cancer cells. TLR9 is a receptor for microbial DNA. Due to the anatomic localization of the prostate gland, whether normal or cancer-containing, it is susceptible to ascending infections. The possibility that also the natural TLR9-ligand, bacterial DNA, might stimulate invasion in TLR9⁺ prostate cancer cells was investigated. As shown in FIG. 19A, treatment with E. Coli DNA induced a dose-dependent increase in the invasive capacity of PC-3 cells in vitro. Also this effect was inhibited by chloroquine, which inhibits TLR9signaling through interfering with endosomal acidification (FIG. 19B). Taken together, the results indicate that bacterial DNA stimulates prostate cancer invasion via TLR9.

Estradiol upregulates TLR9 expression. The growth of prostate cancer is regulated by sex-hormones. Skeletal changes in the TLR9 knockout mice suggest that TLR9 expression may be regulated via sex steroids. The possibility was investigated that these hormones might regulate TLR9 expression also in prostate cancer cells. To study this, the androgen- and estrogen-receptor expressing LnCaP cells were cultured for 24 h in serum-free conditions in the presence of vehicle or 10⁻⁸ or 10⁻¹¹ M estradiol or testosterone. TLR9 expression was studied in Western blots. As shown in FIG. 20, estradiol but not testosterone up-regulated TLR9 expression in these cells.

A number of embodiments of the invention have been described. Nevertheless, it will be understood that various modifications may be made without departing from the spirit and scope of the invention. For example, breast cancer cells were used but other cancer cells can be used. Similarly, chloroquine was used, but various modifications and changes in chloroquine would work as well. Accordingly, other embodiments are within the scope of the following claims.

Throughout this application, various publications are referenced. The disclosures of these publications in their entireties are hereby incorporated by reference into this application in order to more fully describe the state of the art to which this invention pertains.

1. A method of reducing the invasiveness of a cancer cell in a subject comprising administering to the subject an effective amount of an inhibitor of Toll Like Receptor-9 (TLR9) signaling.

2. The method of claim 1, wherein the cancer cell is a TLR9 expressing cell.

3. The method of claim 1, wherein the inhibitor of TLR9 signaling is an inhibitor of endosomal maturation.

4. The method of claim 3, wherein the inhibitor of endosomal maturation is selected from the group consisting of chloroquine, quinacrine, monesin, bafilomycin A1 and wortmannin.

5. The method of claim 1, wherein the inhibitor of TLR9 signaling is an MMP13 antagonist.

6. The method of claim 1, wherein the inhibitor of TLR9 signaling is a TLR9 antagonist.

7. The method of claim 6, wherein the TLR9 antagonist is a functional nucleic acid or an antibody.

8. The method of claim 1, wherein the inhibitor of TLR9 signaling is a TRAF6 antagonist.

9. The method of claim 1, wherein the cancer cell is selected from the group consisting of astrocytoma, brain cancer cell, breast cancer cell, prostate cancer cell, lung cancer cell, and gastric cancer cell.

10. The method of claim 9, wherein the cancer cell is a breast cancer cell.

11. The method of claim 9, wherein the cancer cell is a prostate cancer cell.

12. The method of claim 1, further comprising identifying the cancer cell as a TLR9 expressing cell.

13. A method of determining whether a cancer cell is invasive, comprising measuring the level of expression or activity of TLR9 in the cancer cell, wherein an increase in the level of expression or activity of TLR9 as compared to control indicates that the cancer cell is invasive.

14. The method of claim 13, wherein the level of TLR9 mRNA is measured.

15. The method of claim 13, wherein the level of TLR9 protein is measured.

16. A method of treating cancer in a subject, comprising the steps of
a) determining whether one or more of the subject's cancer cells are TLR9 expressing; and
b) administering a CpG-motif containing unmethylated oligonucleotide to the subject, if the cancer cells are negative for TLR9 expression.

17. A method of treating cancer in a subject, comprising the steps of:
a) determining whether one or more of the subject's cancer cells are TLR9 expressing cancer cells; and
b) administering a TLR9 antagonist to the subject, if the cancer cells express TLR9.

18. The method of claim 17, wherein the TLR9 antagonist is an inhibitor of endosomal maturation.

19. The method of claim 18, wherein the inhibitor of endosomal maturation is selected from the group consisting of chloroquine, quinacrine, monesin, bafilomycin A1 and wortmannin.

20. The method of claim 17, wherein the TLR9 antagonist is a functional nucleic acid or an antibody.

21. The method of claim 16, wherein the subject has an astrocytoma, a glioblastoma, breast cancer, prostrate cancer, brain cancer, lung cancer or gastric cancer.

22. The method of claim 16, wherein the subject has breast cancer.

23. The method of claim 16, wherein the subject has prostrate cancer.
